(19) United States
(12) Patent Application Publication
(10) Pub. No.: US 2016/0338977 A1
(43) Pub. Date: Nov. 24, 2016

(54) METHODS AND KITS FOR TREATING DEPRESSION

(71) Applicant: Janssen Pharmaceutica NV, Beerse (BE)

(72) Inventors: Jaskaran Singh, San Diego, CA (US); Ivo Caers, Beerse (BE); Ella Daly, Doylestown, PA (US); Wayne Drevets, Newtown, PA (US)

(73) Assignee: Janssen Pharmaceutica NV, Beerse (BE)

(21) Appl. No.: 15/159,257

(22) Filed: May 19, 2016

Related U.S. Application Data
(60) Provisional application No. 62/164,026, filed on May 20, 2015.

(51) Int. Cl. A61K 31/137

(52) U.S. Cl. CPC A61K 31/137

(57) ABSTRACT

The present invention is directed to, inter alia, methods and kits for the treatment of depression (preferably, treatment resistant depression), or for the treatment of depression in a suicidal patient, and/or for the treatment and/or prevention of suicidality (e.g. suicidal ideations) comprising administering esketamine according to certain dosing regimens.

### CROSS REFERENCE TO RELATED APPLICATIONS

This application claims the benefit of U.S. Provisional Application 62/164,026, filed on May 20, 2015, which is incorporated by reference herein in its entirety.

### TECHNICAL FIELD

The present invention is directed to, inter alia, methods and kits for the treatment of depression.

### BACKGROUND

Major Depressive Disorder is defined as the presence of one of more major depressive episodes that are not better accounted for psychotic disorder or bipolar disorder. A major depressive episode is characterized by meeting five or more of the following criteria during the same 2 week period which represent a change in functioning and include at least depressed/sad mood or loss of interest and pleasure, indifference or apathy, or irritability and is usually associated with a change in a number of neurovegetative functions, including sleep patterns, appetite and body weight, motor agitation or retardation, fatigue, impairment in concentration and decision making, feelings of shame or guilt, and thoughts of death or dying (Harrison's Principles of Internal Medicine, 2000). Symptoms of a depressive episode include depressed mood; markedly diminished interest or pleasure in all, or almost all, activities most of the day; weight loss when not dieting or weight gain, or decrease or increase in appetite nearly every day; insomnia or hypersomnia nearly every day; psychomotor agitation or retardation nearly every day; fatigue or loss of energy nearly every day; feelings of worthlessness or excessive or inappropriate guilt nearly every day; diminished ability to think or concentrate, or indecisiveness, nearly every day; recurrent thoughts of death, recurrent suicidal ideation without a specific plan, or a suicide attempt or a specific plan for committing suicide. Further, the symptoms cause clinically significant distress or impairment in social, occupational, or other important areas of functioning. (Diagnostic and Statistical Manual of Mental Disorders, 5^{th} Edition, American Psychiatric Association, 2013)

Current treatment options for unipolar depression include monotherapy or combination therapy (Seroquel® and Abilify® for adjunctive therapy) with various classes of drugs including mono-amine oxidase inhibitors, tricyclic antidepressants, serotonin specific reuptake inhibitors, serotonin noradrenergic reuptake inhibitors, noradrenaline reuptake inhibitors, “natural products” (such as Kava-Kava and St. John's Wort), dietary supplements (such as s-adenosylmethionine), antipsychotics, psychotherapy and others. More specifically, drugs used in the treatment of depression include, but are not limited to imipramine, amitriptyline, desipramine, nortriptyline, doxepin, protriptyline, trimipramine, maprotiline, amoxapine, trazodone, bupropion, chlomipramine, fluoxetine, citalopram, sertraline, paroxetine, tianeptine, nefazadone, venlafaxine, desvenlafaxine, duloxetine, reboxetine, mirtazapine, phenelzine, tranylcypromine, moclobemide, aripiprazole and/or quetiapine fumarate. Several of these agents including, but not limited to, serotonin reuptake inhibitors are also used when depression and anxiety co-exist, such as in anxious depression.

In the clinic, 40-50% of depressed patients who are initially prescribed antidepressant therapy do not experience a remission of depression symptoms. This group typifies level 1 treatment-resistant depression, that is, a failure to demonstrate an “adequate” response to an “adequate” treatment trial (that is, sufficient intensity of treatment for sufficient duration). Moreover, about approximately 30% of depressed patients remain partially or totally treatment-resistant to at least two antidepressant treatments including combination treatments. Increasingly, treatment of treatment-resistant depression includes augmentation strategies including treatment with pharmacological agents such as, antipsychotics (such as quetiapine, aripiprazole, olanzapine, risperidone, and the like), lithium, carbamazepine, and triiodothyronine, and the like; adjunctive electroconvulsive therapy; adjunctive transcranial magnetic stimulation; etc.

Suicide, also known as completed suicide, is the “act of taking one's own life”. See, http://en.wikipedia.org/wiki/Suicide-cite_note-7. Attempted suicide or non-fatal suicidal behavior is self-injury with the desire to end one's life that does not result in death. Suicidal ideation is the medical term for thoughts about or an unusual preoccupation with suicide, or thoughts of ending one's life or not wanting to live anymore but not necessarily taking any active efforts to do so.

The range of suicidal ideation varies greatly from fleeting to chronic and progress to detailed planning, role playing, and unsuccessful attempts, which may be deliberately constructed to fail or be discovered, or may be fully intended to result in death. Although not all who have suicidal ideation go on to make suicide attempts, a significant proportion do. Suicidal ideation is generally associated with depression (at about 60-70% of all cases).

Suicidal ideation which may include, for example, suicidal thoughts, may also include other related signs and symptoms. Some symptoms or co-morbid conditions may include unintentional weight loss, feeling helpless, feeling alone, excessive fatigue, low self-esteem, presence of consistent mania, excessively talkative, intent on previously dormant goals, feel like one's mind is racing. The onset of symptoms like these with an inability to get rid of or cope with their effects, a possible form of psychological inflexibility, is one possible trait associated with suicidal ideation. They may also cause psychological distress, which is another symptom associated with suicidal ideation. Symptoms like these related with psychological inflexibility, recurring patterns, or psychological distress may in some cases lead to the onset of suicidal ideation. Other possible symptoms and warning signs include: hopelessness, anhedonia, insomnia, depression, severe anxiety, angst, impaired concentration, psychomotor agitation, panic attack and severe remorse.

There are also several psychiatric disorders that appear to be comorbid with suicidal ideation or considerably increase the risk of suicidal ideation. The following disorders have been shown to be the strongest predictors of suicidal ideation/disorders in which risk is increased to the greatest extent: major depressive disorder (MDD), dysthymia, bipolar disorder, schizophrenia, and PTSD. The main treatments for suicidality and/or suicidal ideation include: hospitalization, outpatient treatment, and medication. Hospitalization allows the patient to be in a secure, supervised environment to prevent their suicidal ideation from turning into suicide attempts. In most cases, individuals have the freedom to choose which treatment they see fit for themselves. However, there are several circumstances in which individuals can be hospitalized involuntarily, per state law including circumstances where an individual poses danger to self or others and where an individual is unable to care for one's self.

Outpatient treatment allows individuals to remain at their place of residence and receive treatment when needed or on a scheduled basis. Before allowing patients the freedom that comes with outpatient treatment, physicians evaluate several factors of the patient. These factors include the patient's level of social support, impulse control and quality of judgment. After the patient passes the evaluation, they are often asked to consent to a “no-harm contract”. This is a contract formulated by the physician and the family of the patient. Within the contract, the patient agrees not to harm themselves, to continue their visits with the physician, and to contact the physician in times of need. These patients are then checked on routinely to assure they are maintaining their contract and staying out of troublesome activities.

There are limited pharmacological treatment options for those experiencing suicidal ideation in patients with depression. However, none of these treatment options are approved and prescribing medication to treat suicidal ideation can be difficult. One reason for this is because many medications lift patients' energy levels before lifting their mood. This puts them at greater risk of following through with attempting suicide. Therefore, the medication prescribed to one suicidal ideation patient may be completely different than the medication prescribed to another patient. Although research is largely in favor of the use of antidepressants for the treatment of suicidal ideation associated with depression, in some cases antidepressants are claimed to be associated with increased suicidal ideation. Upon the start of using antidepressants, many clinicians will note that sometimes the sudden onset of suicidal ideation may accompany treatment. This has caused the Food and Drug Administration (FDA) to issue a warning stating that sometimes, particularly in adolescents and young adults, the use of antidepressants may actually increase the thoughts of suicidal ideation.

Ketamine (a racemic mixture of the corresponding S- and R-enantiomers) is an NMDA receptor antagonist, with a wide range of effects in humans, depending on the dose, including for example, analgesia, anesthesia, hallucinations, dissociative effects, elevated blood pressure and bronchodilation. Ketamine is primarily used for the induction and maintenance of general anesthesia. Other uses include sedation in intensive care, analgesia (particularly in emergency medicine and treatment of bronchospasms). Ketamine has also been shown to be efficacious in the treatment of depression (also in those who have not responded to other antidepressant treatment). In patients with major depressive disorders, ketamine has additionally been shown to produce a rapid antidepressant effect, acting within hours.

The S-ketamine enantiomer (or S-(+)-ketamine or esketamine) has higher potency or affinity for the NMDA receptor than the R-enantiomer (arketamine), thus potentially allowing for lower dosages than with ketamine; and is available for medical use under the brand name KETANEST in some countries.

There remains a high, unmet medical need to provide an effective and rapidly acting treatment for depression and maintaining that improvement such as could be provided using esketamine.

### SUMMARY OF THE INVENTION

The general description and the following detailed description are exemplary and explanatory only and are not restrictive of the invention, as defined in the appended claims. Other aspects of the present invention will be apparent to those skilled in the art in view of the detailed description of the invention as provided herein.

One aspect of the present invention involves methods of treating depression in a patient comprising administering an effective amount of esketamine to the patient at a given frequency and during an induction phase of defined duration, and administering an effective amount of esketamine to the patient less frequently during a subsequent maintenance phase of defined duration. In certain embodiments, the methods include administering an effective amount of esketamine to the patient during an induction phase and at a frequency of at least once weekly for at least about 1 week, and administering an effective amount of esketamine to the patient during a subsequent maintenance phase and at a frequency of no more often than every other day. In other embodiments, the methods further include adjusting the frequency of administration of said esketamine for said patient. In further embodiments, the methods include administering an effective amount of esketamine to the patient during an induction phase at a frequency of no more often than every other day and administering an effective amount of esketamine to the patient during a subsequent maintenance phase at a frequency of at least once weekly for at least about 1 week. In yet other embodiments, the frequency of administration of esketamine in maintenance phase is less often than the frequency of administration in the induction phase. In still other embodiments, the effective amount is about 10 to about 200 mg.

In another aspect, kits for administering esketamine to a patient in need thereof are provided and include a first dosage unit comprising an effective amount of esketamine for administration to the patient at a given frequency in an induction phase of at least about 1 week, and a second dosage unit comprising an effective amount of esketamine for administration to the patient less frequently than in said induction phase of a defined duration in a maintenance phase. The second dosage unit is administered to the patient after the induction phase. In certain embodiments, the kits include a first dosage unit comprising an effective amount of esketamine for administration to the patient at a frequency at least once weekly in an induction phase of at least about 1 week, and a second dosage unit comprising an effective amount of esketamine for administration to the patient at a frequency of no more often than every other day in a maintenance phase that follows the induction phase. In other embodiments, the kits include a first dosage unit comprising an effective amount of esketamine for administration to the patient at a frequency of no more often than every other day in an induction phase of at least about 1 week, and a second dosage unit comprising an effective amount of esketamine for administration to the patient at a frequency of at least once weekly for at least about 1 week in a maintenance phase that follows the induction phase.

### BRIEF DESCRIPTION OF THE FIGURES

The summary, as well as the following detailed description, is further understood when read in conjunction with the appended drawings. For the purpose of illustrating the invention, there are shown in the drawings exemplary embodiments of the invention; however, the invention is not limited to the specific methods, compositions, and devices disclosed. In addition, the drawings are not necessarily drawn to scale. In the drawings:

FIGS. 1A-1B are line graphs illustrating arithmetic mean (+/−SE) MADRS total score over time. FIG. 1A is data from Period 1 after administration of a placebo (), 28 mg of esketamine (▴), 56 mg of esketamine (▾), or 84 mg of esketamine (▪). FIG. 1B is data from Period 2 for subjects have a QIDS-SR16≧11 at the end of period 1 and were re-randomized.

FIGS. 2A-2B are line graphs illustrating the mean changes (+/−SE) MADRS total score over time. FIG. 1A is data from Period 1 after administration of a placebo (), 28 mg of esketamine (▴), 56 mg of esketamine (▾), or 84 mg of esketamine (▪). FIG. 1B is data from Period 2 for subjects having a QIDS-SR₁₆≧11 at the end of period 1 and were re-randomized.

FIG. 3 is a line graph illustrating the MADRS total score difference from the placebo as a function of dose (14, 28, 56, and 84 mg-◯) for Periods 1 and 2.

FIG. 4 is a mean data plot of Clinician-Assessed Dissociative Symptom Scale (CADSS) total score over time for Periods 1 and 2 after administration of a placebo (), 28 mg of esketamine (▴), 56 mg of esketamine (▾), or 84 mg of esketamine (▪).

### DETAILED DESCRIPTION OF THE INVENTION

In an effort to find efficacious regimens of administering esketamine to patients having depression, the inventors found that the frequency of esketamine administration to a patient was unexpectedly successful in treating a patient. In fact, not only do the methods described herein provide rapid treatment of depression, but they also provide long-term maintenance of individuals having depression, notwithstanding the short half-life of esketamine.

### I. DEFINITIONS

As used herein, the term “esketamine” is the (S)-enantiomer of ketamine and, as its corresponding hydrochloride salt, has the following structure and is known as (S)-2-(2-chlorophenyl)-2-(methylamino)cyclohexanone hydrochloride.

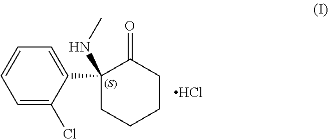

As used herein, the term “depression” includes major depressive disorder, unipolar depression, treatment resistant depression, depression with anxious distress, bipolar depression and dysthymia (also referred to as dysthymic disorder). In one embodiment, the depression is major depressive disorder, unipolar depression, treatment resistant depression and bipolar depression. In another embodiment, the depression is treatment-resistant depression.

The methods described herein are also useful in the treatment of depression in suicidal patients. One skilled in the art will recognize that the term “depression in suicidal patients” includes depression when diagnosed in a patient that also exhibits at least one symptom of suicidality, for example suicidal ideations and/or behaviors (e.g. intent, planning, etc.). Thus, “depression in suicidal patients” includes, but is not limited to, major depressive disorder in suicidal patients, unipolar depression in suicidal patients, treatment resistant depression in suicidal patients, depression with anxious distress in suicidal patients, bipolar depression in suicidal patients and dysthymia in suicidal patients.

As used herein, the term “treatment-refractory or treatment-resistant depression” and the abbreviation “TRD” shall be defined as a major depressive disorder that does not respond to a least two antidepressant regimens, treatments, drugs, therapy, or any combinations thereof.

Some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

As used herein, unless otherwise noted, the terms “treating”, “treatment” and the like, shall include the management and care of a subject or patient (preferably mammal, more preferably human) for the purpose of combating a disease, condition, or disorder and include the administration of esketamine to prevent the onset of the symptoms or complications, alleviate the symptoms or complications, or eliminate the disease, condition, or disorder. Similarly, “treatment” is used to encompass (a) reduction in the frequency of one or more symptoms; (b) reduction in the severity of one or more symptoms; (c) the delay or avoidance of the development of additional symptoms; and/or (d) delay or avoidance of the development of the disorder or condition, or any combination thereof.

As used herein, unless otherwise noted, the terms “subject” and “patient” may be used interchangeably and refer to an animal, preferably a mammal, most preferably a human, who has been the object of treatment, observation or experiment. Preferably, the subject or patient has experienced and/or exhibited at least one symptom of the disease or disorder to be treated and/or prevented. One skilled in the art will further recognize that the methods of treatment and kits are directed to subjects or patients in need of such treatment, prevention or dosing regimen, more particularly to subjects or patients diagnosed with or exhibiting at least one symptom of depression (preferably, meeting the criteria for major depressive disorder or episode) regardless of type or underlying cause. In one embodiment, the subject or patient has been diagnosed with or exhibits at least one symptom of depression (preferably, meeting the criteria for major depressive disorder or episode) and who has further been diagnosed with or exhibits at least one symptom of suicidality (e.g. suicidal ideations, behaviors, and/or intent).

Further, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein.

The present invention may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. The term “plurality,” as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

### II. METHODS OF USING ESKETAMINE

As noted above, methods of treating depression in a patient are described. The methods include administering esketamine in at least two phases, i.e., an induction phase and a maintenance phase. Accordingly, an effective amount of esketamine is administered in each phase. The effective amount of esketamine may be the same in each phase or may differ. The methods described herein permit optimizing dosages of esketamine for administration to a patient having or being predisposed to depression. Advantageously, the methods described herein do not require adjustment of the esketamine dosage. In fact, esketamine may be administered during the phases discussed herein (e.g., induction, optimization, and maintenance) at the lowest dosing frequency at which an esketamine response is observed and maintained in a patient.

Therapeutically effective amounts for esketamine include amounts that elicit the biological or medicinal response in a tissue system, animal or human that is being sought by a researcher, veterinarian, medical doctor or other clinician, which includes alleviation of the symptoms of the disease or disorder being treated. Optimal dosages to be administered may be readily determined by those skilled in the art, and may vary with the mode of administration, the strength of the preparation and the advancement of the disease condition. Such factors including the particular patient being treated, including patient's sex, age, weight, diet, time of administration and concomitant diseases, among others. Therapeutic dosage amounts and regimens for esketamine approved for sale are publicly available, for example as listed on packaging labels, in standard dosage guidelines, in standard dosage references such as the Physician's Desk Reference (Medical Economics Company or online at http://www.pdrel.com) or other sources. In certain embodiments, the effective amount of each dose of esketamine in the induction phase is about 1 mg to about 200 mg. In other embodiments, the effective amount in the induction phase is about 10 to about 100 mg. In further embodiments, the effective amount in the induction phase is about 28 mg to about 84 mg. In yet other embodiments, the effective amount of esketamine in the induction phase is about 56 mg to about 84 mg. In other embodiments, the effective amount esketamine in the induction phase is about 56 mg. In further embodiments, the effective amount esketamine in the induction phase is about 84 mg.

The induction phase preferably is sufficiently long as to achieve a robust, stable reduction of depressive symptoms, and depends on factors including, without limitation, the particular patient and/or the patient's sex, age, weight, diet, time of administration, administration frequency and concomitant diseases. In certain embodiments, the induction phase includes a period of about 1 to 12 weeks. In other embodiments, the induction phase includes a period of about 2 to about 8 weeks. In further embodiments, the induction phase is a period of about 2 to about 6 weeks. In other embodiments, the induction phase is a period of about 1 week. In still further embodiments, the induction phase is a period of about 2 weeks. In yet other embodiments, the induction phase is a period of about 3 weeks or a period of about 4 weeks.

Esketamine is administered at least once weekly to the patient during the induction phase. The specific days during the week during which esketamine may be administered may vary depending on factors including, without limitation, the patient's sex, age, weight, diet, time of administration, dose administered and concomitant diseases. In some embodiments, esketamine is administered at least twice weekly to the patient during the induction phase. In other embodiments, esketamine is administered at least three times a week to the patient during the induction phase. In other embodiments, the second dose of esketamine during a given week is administered at least 2 days after the first dose. In other embodiments, the second dose of esketamine is administered 3 days after the first dose. In further embodiments, the second dose of esketamine is administered 4 days after the first dose. In yet other embodiments, esketamine is administered at a frequency of no more often than every other day during the induction phase.

The induction phase is a period of at least about 1 week. In other embodiments, the induction phase is a period of at least about 2 weeks. In still further embodiments, the induction phase is a period of at least 3 weeks. In other embodiments, the induction phase is a period of at least 4 weeks. In yet further embodiments, the induction phase is a period of about 1 week to about 12 weeks.

The particular route of esketamine administration during the induction phase depends on a number of factors as determined by one skilled in the art. Esketamine can, for example, be administered non-orally during the induction phase. For example, it can be administered intravenously, intranasally, intramuscularly, subcutaneously, transdermally, buccally, or rectally during the induction phase. Preferably, esketamine is administered intranasally during the induction phase. Esketamine can also be administered orally during the induction phase. For example, it can be administered orally, i.e., liquid, suspension, caplet, tablet, tablet-in-capsule, capsule, or dissolving film, or sublingually during the induction phase. In certain embodiments, a single route of administration may contain the required effective amount of esketamine. In some embodiments, 2 or more doses of the same route of administration are administered in order to administer the effective amount of esketamine. In other embodiments, 1 or more dose of one route of administration and 1 or more dose of another route of administration are administered in order to administer the effective amount of esketamine. For example, it may be necessary to spray the intranasal administration device 2 or more times; ingest 2 or more tablets, caplets, tablet-in-capsules, capsules, or dissolving films; apply 2 or more suppositories; or inject 2 or more liquids to administer the effective amount of esketamine.

The methods described herein also include a maintenance phase. One skilled in the art will recognize that the maintenance phase described herein may continue until further treatment is not required and as indicated by, for example, prolonged remission of the depression (including for example, the remission of one or more symptoms associated with depression), social and/or occupational functional improvement to normal or premorbid levels, or other known measures of depression.

An effective amount of esketamine is administered to the patient during the maintenance phase. As noted above, the amount of esketamine administered during the maintenance phase is an amount that elicits the biological or medicinal response in a tissue system discussed above for the induction phase. In certain embodiments, the effective amount of esketamine is the amount which maintains a pharmacodynamic steady state of esketamine attained in the induction phase.

Optimal dosages to be administered may be readily determined by those skilled in the art, and may vary with such factors as the mode of administration, the advancement of the disease condition, patient's sex, age, weight, diet, time of administration and concomitant diseases, results of the induction phase, among others. In certain embodiment, the effective amount of each dose of esketamine is about 1 to about 200 mg. In other embodiments, the effective amount in the maintenance phase is about 10 to about 100 mg. In further embodiments, the effective amount in the maintenance phase is about 28 mg to about 84 mg. In yet other embodiments, the effective amount of esketamine in the maintenance phase is about 56 mg to about 84 mg. In other embodiments, the effective amount esketamine in the maintenance phase is about 56 mg. In further embodiments, the effective amount esketamine in the maintenance phase is about 84 mg.

During the maintenance phase, esketamine preferably is administered less frequently than the frequency in the induction phase. In some embodiments, esketamine is administered during the maintenance phase at a frequency of no more often than every other day. In certain embodiments, esketamine is administered during the maintenance phase twice weekly. In other embodiments, esketamine is administered during the maintenance phase three times a week. In yet other embodiments, esketamine is administered at a frequency of at least once weekly during the maintenance phase. In further embodiments, esketamine is administered during the maintenance phase every other week or frequencies there between. In yet other embodiments, esketamine is administered during the maintenance phase every three weeks or frequencies there between. In still further embodiments, esketamine is administering during the maintenance phase once a month or frequencies there between.

The frequency of administration of esketamine during the maintenance phase may be the same as the frequency of administration of esketamine during the induction phase. Alternatively, the frequency of administration during the maintenance phase may differ from the frequency of administration of esketamine during the induction phase. In other embodiments, the frequency of administration of esketamine is reduced in the maintenance phase.

The particular route of esketamine administration during the maintenance phase depends on a number of factors as determined by one skilled in the art. The esketamine, for example, can be administered by the patient during the maintenance phase using a route that does not involve hospital intervention. Esketamine can be administered orally, intravenously, intranasally, intramuscularly, subcutaneously, sublingually, transdermally, or rectally during the maintenance phase. In another embodiment, esketamine is administered orally during the maintenance phase. For example, it can be administered orally, i.e., liquid, suspension, caplet, tablet, tablet-in-capsule, capsule, or dissolving film, or sublingually during the maintenance phase.

Frequency adjustment of esketamine administration can be accomplished by a one-time switch in frequency or may be determined over two or more administrations of esketamine. By doing so, the attending physician or the like may determine an optimal frequency for administration of esketamine and thereby tailor the administration to the patient. This adjustment may be accomplished during the maintenance phase, thereby permitting adjusting the frequency of esketamine administration to the patient. The esketamine frequency may be adjusted at any point during the maintenance phase.

Also contemplated by these methods is the administration of rescue doses of esketamine. The term “rescue dose” as used herein refers to one or more additional doses of esketamine in addition to the regularly prescribed dose. The rescue dose may be administered during the induction phase, maintenance phase, or any combination thereof. The amount of esketamine in the rescue dose may be determined by the prescribing physician or clinician and will depend on any of the factors discussed herein. In certain embodiments, the rescue dose of esketamine is the same as the effective dose used during the induction and/or maintenance phase. In other embodiments, the rescue dose differs from the effective dose used during the induction and/or maintenance phase.

One skilled in the art will recognize that in the methods and kits described herein, the maintenance of the esketamine response in a patient may be determined by for example, a clinician, physician, psychiatrist, psychologist, or other suitable medical professional. Additionally, maintenance of the antidepressant response may be established by for example, an absence of relapse of the depression (or one or more symptoms of the depression), an absence of the need for additional or alternate treatment(s) for the depression, an absence of the worsening of the depression, an absence of the need for hospitalization for a suicidal attempt or to prevent suicide. The physician or attending clinician may utilize any technique known in the art including, without limitation, general patient evaluation, diagnostic questionnaires, and evaluations such as the Clinical Global Impression—Severity (CGI-S) scale, EuroQol; 5 dimension; 5 level (EQ-5D-5L), Columbia Suicide Severity Rating Scale (C-SSRS), Patient Health Questionnaire-9 Item (PHQ-9), Sheehan Disability Scale (SDS), Inventory of Depressive Symptomatology-Clinician rated, 30-item scale (IDS-C₃₀), Montgomery-Åsberg Depression Rating Scale (MADRS) questionnaire, Hamilton rating scale for depression (HAM-D or HDRS) Beck Scale for Depression, or Quick Inventory of Depressive Symptomology (QIDS). The esketamine frequency may be evaluated and/or changed if the score from one or more of the above-noted scales or questionnaire changes.

One skilled in the art will recognize that wherein methods of prevention are described, a subject in need of thereof (i.e. a subject in need of prevention) shall include any subject or patient (preferably a mammal, more preferably a human) who has experienced or exhibited at least one symptom of the disorder, disease or condition to be prevented. Further, a subject in need thereof may additionally be a subject (preferably a mammal, more preferably a human) who has not exhibited any symptoms of the disorder, disease or condition to be prevented, but who has been deemed by a physician, clinician or other medical profession to be at risk of developing said disorder, disease or condition. For example, the subject may be deemed at risk of having new episodes of depression (and therefore in need of secondary prevention or preventive treatment) as a consequence of the subject's medical history, including, but not limited to, family history, pre-disposition, co-existing (comorbid) disorders or conditions, genetic testing, and the like.

One skilled in the art will recognize that, both in vivo and in vitro trials using suitable, known and generally accepted cell and/or animal models are predictive of the ability of a test compound to treat or prevent a given disorder. One skilled in the art will further recognize that human clinical trials including first-in-human, dose ranging and efficacy trials, in healthy patients and/or those suffering from a given disorder, may be completed according to methods well known in the clinical and medical arts.

### III. KITS

Also described herein are kits for administering esketamine to a patient in need thereof. The representative kits include one or more dosage units comprising an effective amount of esketamine for administration to a patient and at a given frequency at least about 1 week in an induction phase. The kits also includes a second dosage unit comprising an effective amount of esketamine for administration to the patient and at a frequency less than in the induction phase for a defined duration in a maintenance phase. The second dosage unit preferably is administered to the patient after the induction phase.

The kit may contain one to about 8 dosage units for administration of the required dosage at the specific period during the induction phase. For example, the kit may contain 2 dosage units, each for administration to the patient sequentially in order to obtain the first required dosage of esketamine in the induction phase. In certain embodiments, the kit contains one dosage unit for administration to the patient during the induction phase. In other embodiments, the kit contains two dosage units for administration to the patient during the induction phase. In further embodiments, the kit contains three dosage units for administration to the patient during the induction phase. In yet other embodiments, the kit contains four dosage units for administration to the patient during the induction phase. In still further embodiments, the kit contains five dosage units for administration to the patient during the induction phase. In other embodiments, the kit contains six dosage units for administration to the patient during the induction phase. In further embodiments, the kit contains seven or eight dosage units for administration to the patient during the induction phase.

The dosage unit for administration during the induction phase may be formulated for delivery by any non-oral means including, for example, intravenously, intranasally, intramuscularly, subcutaneously, transdermally, or rectally. The first dosage unit preferably is formulated for intranasal delivery. In other embodiments, the first dosage unit may be formulated for oral means. Such oral means include, without limitation, liquid, suspension, caplet, tablet, tablet-in-capsule, capsule, or dissolving film, or sublingually.

The second dosage unit may be formulated for delivery by any means. In certain embodiments, the second dosage unit is formulated for oral, intravenous, intranasal, intramuscular, subcutaneous, sublingual, transdermal, otic or rectal delivery. In a certain embodiments, it is preferred that the second dosage unit be formulated for oral delivery.

The dosage unit utilized in the induction and/or maintenance phase may be formulated to contain any amount of esketamine, depending on the route of administration. Accordingly, each dosage unit may comprise the required dosage for the patient or may comprise a portion of the esketamine which is required for a single dosage. In certain embodiments, each dosage unit of esketamine contains about 25 to about 100 mg of esketamine. In other embodiments, each dosage unit contains about 25 to about 30 mg of esketamine. In further embodiments, each dosage unit contains about 28 mg of esketamine. In cases where an intranasal spray is used to deliver the esketamine, each spray may be actuated. In certain embodiments, each spray delivered from the intranasal device delivers the entire dosage of esketamine. In other embodiments, each spray delivered from the intranasal device delivers less than the entire dosage of esketamine. In further embodiments, each spray delivered from the intranasal device delivers about half of the dosage of esketamine.

Also optionally included in the kits is a depression symptom rating scale questionnaire. The questionnaire may be for use by the patient alone or in combination with a physician. The questionnaire may be useful for determining the level of depression of the patent at any stage of esketamine administration. In one embodiment, the questionnaire is one or more of the questionnaires noted above.

Instructions for performing the claimed methods and administering esketamine may also be included in the kits described herein.

The kits may be organized to indicate a single formulation containing esketamine or combination of formulations, each containing esketamine. The composition may be sub-divided to contain appropriate quantities of esketamine. The unit dosage can be packaged compositions such as packeted powders, vials, ampoules, prefilled syringes, tablets, caplets, capsules, or sachets containing liquids.

The esketamine may be a single dose or for continuous or periodic discontinuous administration. For continuous administration, a kit may include esketamine in each dosage unit. When varying concentrations of esketamine, the components of the composition containing esketamine, or relative ratios of esketamine or other agents within a composition over time is desired, a kit may contain a sequence of dosage units.

The kit may contain packaging or a container with esketamine formulated for the desired delivery route. The kit may also contain dosing instructions, an insert regarding esketamine, instructions for monitoring circulating levels of esketamine, or combinations thereof. Materials for using esketamine may further be included and include, without limitation, reagents, well plates, containers, markers or labels, and the like. Such kits may be packaged in a manner suitable for treatment of a desired indication

Other suitable components to include in such kits will be readily apparent to one of skill in the art, taking into consideration the desired indication and the delivery route. The kits also may include, or be packaged with, instruments for assisting with the injection/administration of esketamine to the patient. Such instruments include, without limitation, an inhalant, syringe, pipette, forceps, measuring spoon, eye dropper or any such medically approved delivery means. Other instrumentation may include a device that permits reading or monitoring reactions in vitro.

Esketamine may be provided in dried, lyophilized, or liquid forms. When reagents or components are provided as a dried form, reconstitution generally is by the addition of a solvent. The solvent may be provided in another packaging means and may be selected by one skilled in the art.

A number of packages or kits are known to those skilled in the art for dispensing pharmaceutical agents. In certain embodiments, the package is a labeled blister package, dial dispenser package, or bottle.

Methods for optimizing a dosage of esketamine for a patient having or being predisposed to depression also are provided. These methods can include (a) administering an effective amount of esketamine to the patient during an induction phase at a given frequency of a defined duration, (b) analyzing the effects of the esketamine in the induction phase by rating the depression of the subject, and (c) administering an effective amount of esketamine to the patient during a maintenance phase less frequently of a defined duration.

The following Examples are set forth to aid in the understanding of the invention, and are not intended and should not be construed to limit in any way the invention set forth in the claims which follow thereafter.

### IV. EXAMPLES

The subjects discussed in Examples 1 to 5 included adult men and women, 18 (or older if the minimum legal age of consent in the country in which the study is taking place is >18) to 64 years of age (inclusive), who meet diagnostic criteria for recurrent MDD or single-episode MDD, based upon clinical assessment.

A response is defined as a ≧50% reduction in the initial symptom score, and remission is defined as a total score of ≦12 on the MADRS. At the end of the open-label induction phase, subjects who meet this criterion for response (i.e., ≧50% reduction in the initial symptom score) were considered responders and are eligible to continue treatment after the induction phase.

### Example 1

### Intranasal Induction Phase

This example was a 2-panel, doubly-randomized, double-blind, placebo-controlled, multicenter study. In both panels, each subject participated in a screening phase of up to 4 weeks and/or a double-blind treatment phase which includes two 1-week periods (Period 1 and Period 2).

Sixty-seven (67) subjects with Treatment Resistant Depression (TRD) were randomly assigned in a 3:1:1:1 ratio to 1 of the 4 treatment groups in Table 1.

TABLE 1

Period

| | |
|---|---|
| 1 | 2 |

| | |
|---|---|
| Group | Number of Participants |

| | | |
|---|---|---|
| Placebo | 33 | 6 |
| 28 mg of Esketamine | 11 | 8 |
| 56 mg of Esketamine | 11 | 9 |
| 84 mg of Esketamine | 12 | 5 |

All subjects self-administered the intranasal esketamine (28, 56, or 84 mg) or placebo on non-consecutive days. Each intranasal device contains 2 sprays and contain esketamine deliver 14 mg per spray, for a total of 28 mg per individual device (i.e., 2 sprays). Intranasal treatment sessions did not take place on consecutive days. Food was restricted for 8 hours before each administration of study drug and the drinking of fluids was restricted for at least 30 minutes before the first nasal spray.

TABLE 2

| Intranasal | Time of Administration^{a} | | |
|---|---|---|---|
| Treatment | 0^{a} | 5 minutes | 10 minutes |
| Intranasal Device | First | Second | Third |
| Esketamine | 1 spray of | 1 spray of esketamine | No device |
| 56 mg | esketamine | to each nostril | required |
| | to each nostril | | |
| Esketamine | 1 spray of | 1 spray of esketamine | 1 spray of |
| 84 mg | esketamine | to each nostril | esketamine |
| | to each nostril | | to each |
| | | | nostril |

^{a}Time 0 = time of administration of the first intranasal spray to one nostril from the first intranasal device.

In Period 2, 28 placebo subjects who were eligible for re-randomization at the end of Period 1 were randomly assigned to as noted in Table 1 in a 1:1:1:1 ratio. Subjects eligible for re-randomization had to have a QIDS-SR₁₆ total score >11 at the end of Period 1. Central randomization was implemented in this study. Both randomizations were balanced using randomly permuted blocks and were stratified by study center. The randomization for Period 2 was also stratified by QIDS-SR₁₆ score at the end of Period 1 (moderate=11-16; severe >16) Subjects who were randomly assigned to esketamine in Period 1 continued to receive esketamine in Period 2, while Period 1 placebo subjects with a QIDS-SR₁₆ total score <11 continued with placebo in Period 2.

The Period 1 ITT analysis set consisted of all randomized subjects who received at least 1 dose of study medication during Period 1 and had both the baseline and at least one post baseline value for the primary endpoint measure, MADRS total score, within Period 1. The Period 2 ITT analysis set consisted of all Period 1 placebo subjects who were eligible for re-randomization (QIDS-SR₁₆≧11 at end of Period 1), who actually got re-randomized into Period 2 and received at least 1 dose of study medication during Period 2, and had both the baseline and at least one post baseline MADRS total score within Period 2.

The MADRS consisted of 10 items that covered all of the core depressive symptoms (apparent sadness, reported sadness, inner tension, sleep, appetite, concentration, lassitude, interest level, pessimistic thoughts, and suicidal thoughts). Each item was scored from 0 (item is not present or is normal) to 6 (severe or continuous presence of the symptom). A total score (0 to 60) was calculated by summing the scores of all 10 items. A higher score represented a more severe condition. Sustained response (onset of clinical response) was defined as at least 50% improvement from baseline in the MADRS total score with an onset by Day 2 that was maintained to study Day 15. The primary efficacy endpoint was the change in MADRS total score from baseline to Day 8 in each period, with the analysis based on data combined across both periods. The primary efficacy analysis was performed on the intent-to-treat (ITT) analysis sets that were defined for each period, which included all randomized subjects who received at least 1 dose of study medication during that period and have both the baseline and at least one post baseline MADRS total score within that period. Significance was based on a one-sided alpha level of 0.05.

A separate ANCOVA model was performed on last observation carried forward (LOCF) data for each period. Changes from baseline in Period 1 were modeled with treatment as a factor and Period 1 baseline value as a covariate. Changes from baseline in Period 2 were modeled with treatment, Period 2 baseline QIDS-SR₁₆ score (moderate or severe) as factors and Period 2 baseline value as a covariate. The overall comparison between each esketamine dose and placebo at Day 8 was carried out by a combined test based on the weighted test statistics corresponding to the two esketamine-placebo differences. The weights used were 0.5985084 for Period 1 and 0.4014916 for Period 2.

Periods 1 and 2 result for the MADRS total score favored esketamine, with the least square mean differences from placebo in Table 3.

TABLE 3

Least Square Mean
Difference from Placebo

| Esketamine Dosage (mg) | Period 1 | Period 2 |
|---|---|---|
| 28 | −5.0 | −3.1 |
| 56 | −7.6 | −4.4 |
| 84 | −10.5 | −6.9 |

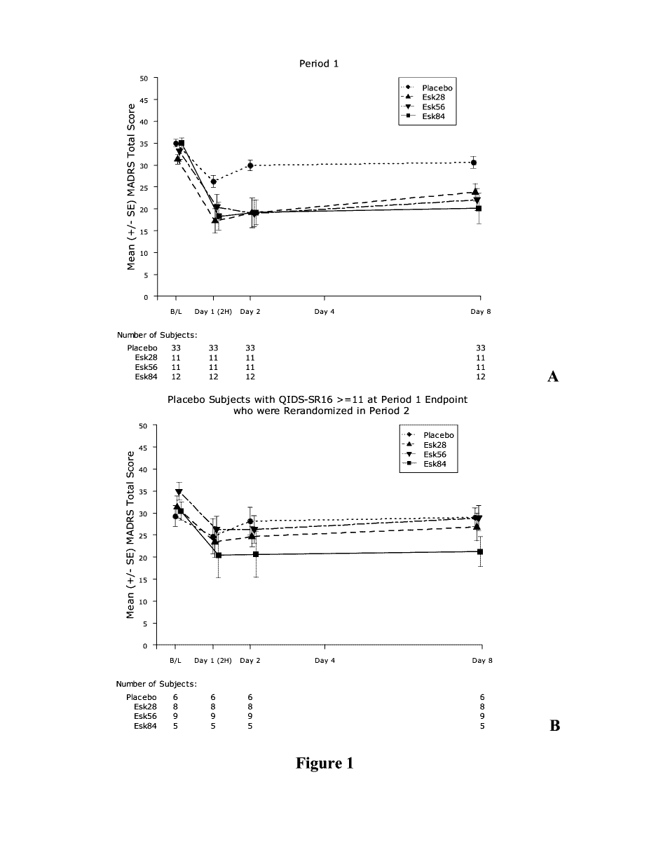

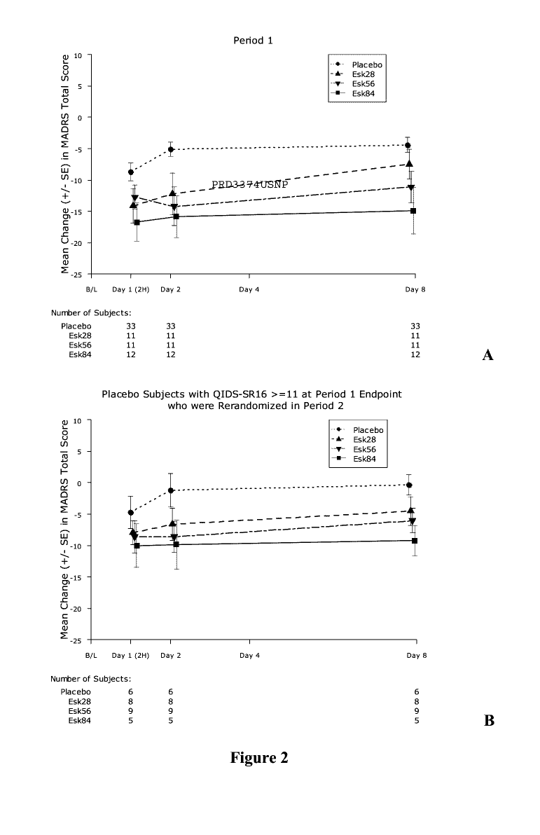

The results of the consistency test show that the results for Period 1 were highly consistent with Period 2 as the upper limit of the confidence intervals for the p-values were ≦0.05 for all three esketamine groups. FIG. 1 shows the arithmetic means (+/−SE) for the MADRS total score over time by period and FIG. 2 shows the mean changes (+/−SE) from the respective period's baseline over time by period. The analysis of Period 1 and Period 2 combined using the weighted combination test, shows that all three esketamine groups were statistically superior to placebo using a one-sided 0.05 significance level. See, Table 4. The mean differences from placebo are in Table 3 and the differences represent the difference from placebo on Day 8 of treatment and were estimated using data from both Periods 1 and 2.

TABLE 4

| Esketamine Dosage (mg) | p Value | Mean Differences (SE) |
|---|---|---|
| 28 | 0.021 | −4.2 (2.09) |
| 56 | 0.001 | −6.3 (2.07) |
| 84 | <0.001 | −9.0 (2.13) |

Results of the sensitivity analysis using a mixed-effects model using repeated measures (MMRM) on observed case data for each period that included factors for time (Day 1, 2 and 8), treatment, country, Period 2 baseline QIDS-SR₁₆ total score [(moderate or severe) included for Period 2 model only], time-by treatment interaction and baseline value (for each respective period) as a covariate were consistent with the ANCOVA analysis. See, Table 5. This analysis used the same weights defined above for the ANCOVA analysis.

TABLE 5

| Esketamine Dosage (mg) | p Value |
|---|---|
| 28 | 0.021 |
| 56 | 0.001 |
| 84 | <0.001 |

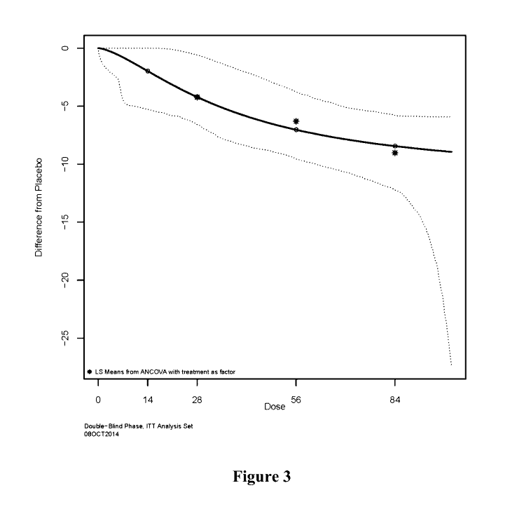

Additional dose response analysis for each period was the estimation of the best fit sigmoidal Eₘₐₓ model based on the observed study efficacy data using the ITT set, and a bootstrapping technique for the calculation of 90% confidence intervals around the estimated treatment effect for a pre-specified set of doses, including the study doses. The results are presented in the best fit sigmoidal Eₘₐₓ curve is displayed in FIG. 3. Although esketamine 14 mg was not included as a dose in Panel A, the dose response model allows for an estimate for this dose. The estimated differences from placebo are in Table 6. As the 90% CI for the treatment difference from placebo included 0 for the esketamine 14 mg dose, this dose is not statistically different from placebo. Esketamine 28 mg, 56 mg and 84 mg were statistically superior to placebo.

TABLE 6

| | Estimated |
|---|---|
| Esketamine Dosage (mg) | Differences |
| | −1.98 |
| 28 | −4.22 |
| 56 | −7.04 |
| 84 | −8.45 |

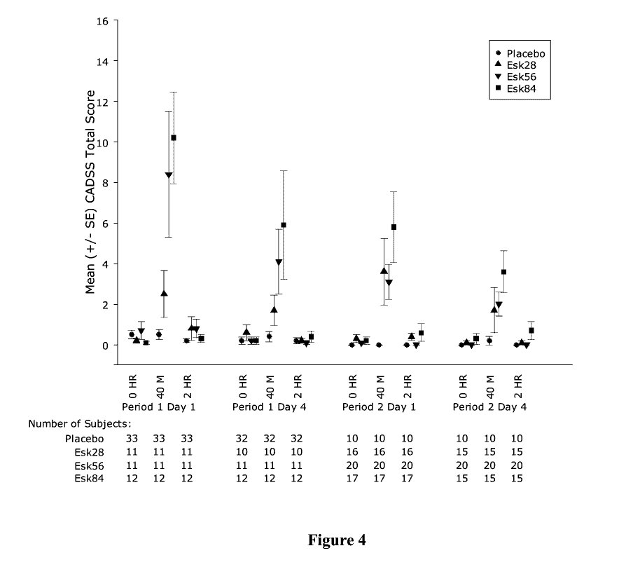

The Clinician Administered Dissociative States Scale (CADSS) was measured prior to the start of each dose, at 40 minutes and at 2 hours post dose. The CADSS is used to assess treatment emergent dissociative symptoms and perceptual changes and the total score ranges from 0 to 92 with a higher score representing a more severe condition. The dissociative and perceptual change symptoms measured by the CADSS, suggest these symptoms had an onset shortly after the start of the dose, with a peak by 40 minutes and resolved by 2 hours post dose. See, FIG. 4.

### Example 2

### Side Effects

The treatment emergent adverse event (TEAE) side effects for the subjects of Example were obtained. See, Table 7. Overall, about 79% of the subjects who received esketamine in either Period 1 or Period 2 of Example 1 experienced at least one TEAE. The most common TEAEs were dizziness, dissociation, headache, dysgeusia, nasal discomfort, nausea, hypoaesthesia oral, dissociative disorder, tunnel vision, oropharyngeal pain, throat irritation, blurred vision, hypersomnia, feeling abnormal, hypertension, insomnia and sedation.

TABLE 7

Patients Experiencing
on TEAE

| Esketamine Dosage (mg) | Period 1 | Period 2 |
|---|---|---|
| Placebo | 52 | 33 |
| 28 | 73 | 38 |
| 56 | 82 | 78 |
| 84 | 83 | 100 |

### Example 3

### Intranasal Optimization Phase

In this example, subjects from Example 1 continue to receive the same intranasal treatment (esketamine) from the induction phase for the 12 weeks of this phase. Each intranasal device contains 2 sprays. The intranasal devices containing esketamine deliver 14 mg per spray, for a total of 28 mg per individual device (i.e., 2 sprays) to administer either 56 or 84 mg per session, i.e. 2 to 3 devices. The intranasal treatment session frequency will be reduced from that in the induction phase (twice weekly) to weekly for the first 4 weeks of this phase (weeks 5-8). Table 8 provides an administration intranasal esketamine schedule for the adjustment phase.

TABLE 8

| | Time of Administration | | |
|---|---|---|---|
| | 0^{a} | 5 minutes | 10 minutes |
| | Device | | |
| | First | Second | Third |
| Placebo | 1 spray of placebo | 1 spray of placebo | 1 spray of placebo |
| | to each nostril | to each nostril | to each nostril |
| Esketamine 56 mg | 1 spray of esketamine | 1 spray of esketamine | 1 spray of placebo |
| | to each nostril | to each nostril | to each nostril |
| Esketamine 84 mg | 1 spray of esketamine | 1 spray of esketamine | 1 spray of esketamine |
| | to each nostril | to each nostril | to each nostril |

After the first 4 weeks, the frequency of intranasal treatment sessions will be reduced to once weekly and further on individualized to once every week or once every other week based on the severity of depression at the end of the dosing interval. This may be assessed using may scales as described herein. In embodiment, this was assessed by the MADRS total score, according to the following:
(i) If the MADRS total score is ≦12 at Week 8, the subject will reduce the frequency to receive intranasal treatment sessions every other week, i.e., next dose at Week 10.
(ii) If the MADRS total score is >12 at any time after Week 8, the frequency of intranasal treatment sessions will be changed back to weekly for the remainder of the adjustment phase.
(iii) Subjects with a MADRS total score >12 at Week 8 or any subsequent time receive weekly intranasal treatment sessions for the remainder of the adjustment phase.

### Example 4

### Intranasal Maintenance Phase

Subjects from Example 3 and in stable remission (MADRS total score for the last 4 weeks of the adjustment phase) after treatment with intranasal esketamine will receive double-blind intranasal treatments. See, Table 9 for the administration of intranasal esketamine in the maintenance phase. Each intranasal device contains 2 sprays and the intranasal devices containing esketamine deliver 14 mg per spray, for a total of 28 mg per individual device (i.e., 2 sprays).

TABLE 9

| | Time of Administration | | |
|---|---|---|---|
| | 0^{a} | 5 minutes | 10 minutes |
| | Device | | |
| | First | Second | Third |
| Placebo | 1 spray of placebo | 1 spray of placebo | 1 spray of placebo |
| | to each nostril | to each nostril | to each nostril |
| Esketamine 56 mg | 1 spray of esketamine | 1 spray of esketamine | 1 spray of placebo |
| | to each nostril | to each nostril | to each nostril |
| Esketamine 84 mg | 1 spray of esketamine | 1 spray of esketamine | 1 spray of esketamine |
| | to each nostril | to each nostril | to each nostril |

At the start of this phase, subjects receiving intranasal treatment sessions on a weekly basis will stay at the same weekly intranasal treatment session frequency for at least the first 4 weeks of this phase. After the first 4 weeks of this phase and for the duration of the phase, the intranasal treatment session frequency will be adjusted (if applicable) based on the guidance below. The MADRS score will be assessed weekly and changes to the intranasal treatment session frequency will be based on the MADRS total score according to the following:
(i) If the MADRS total score ≦12 and the frequency is weekly, the frequency will be changed to every other week.
(ii) If the MADRS total score >12 and the frequency is weekly, no change in frequency.
(iii) If the MADRS total score >12 and the frequency is every other week, the frequency will be changed to weekly

### Example 5

### Oral Antidepressant Study

This example is a randomized, double-blind, parallel-group, active-controlled, multicenter study to evaluate the efficacy, safety, and tolerability of intranasal esketamine plus an oral antidepressant compared with an oral antidepressant (active comparator) plus intranasal placebo in delaying relapse of depressive symptoms in adult men and women with TRD who are in stable remission after an induction and adjustment course with intranasal esketamine plus an oral antidepressant. The study has 5 phases:
Phase 1: 4-week screening/prospective observational phase, with an optional taper of up to 3 weeks for oral antidepressant medication
Phase 2: 4-week open-label induction phase
Phase 3: A 12-week optimization phase
Phase 4: A variable duration maintenance phase
Phase 5: A 2-week follow-up phase

A. Induction Phase:

Subjects will receive intranasal esketamine (flexible dose: 56 mg or 84 mg) treatment sessions twice weekly for 4 weeks. One device will be used at each time point. Each intranasal device contains 2 sprays. The intranasal devices containing esketamine deliver 14 mg per spray, for a total of 28 mg per individual device (i.e., 2 sprays). See, Table 10.

TABLE 10

| | Time of Administration^{a} | | |
|---|---|---|---|
| | 0^{a} | 5 minutes | 10 minutes |
| Intranasal | Intranasal Device | | |
| Treatment | First | Second | Third |
| Esketamine 56 mg | 1 spray of esketamine | 1 spray of esketamine | No device required |
| | to each nostril | to each nostril | |
| Esketamine 84 mg | 1 spray of esketamine | 1 spray of esketamine | 1 spray of esketamine |
| | to each nostril | to each nostril | to each nostril |

In addition, all subjects will administer an oral antidepressant The assigned oral antidepressant will be 1 of 4 oral antidepressant medications (duloxetine, escitalopram, sertraline, or venlafaxine extended release [XR]), that the subject has not previously had a nonresponse to in the current depressive episode, has not been previously intolerant to (lifetime). Dosing of the oral antidepressant will begin on Day 1 and be daily with a forced titration to the maximally tolerated dosage to ensure that the oral antidepressant is taken at an adequate dosage and duration. If higher doses are not tolerated, a down-titration may be performed. The subject's maximum tolerated dose is not lower than the following minimum therapeutic doses: sertraline (50 mg/day), venlafaxine XR (150 mg/day), escitalopram (10 mg/day), and duloxetine (60 mg/day).

B. Optimization Phase

At the end of the induction phase, subjects who respond (have a 50% reduction in the MADRS total score from baseline (Day 1 prior to the first intranasal dose) to the end of the 4-week induction phase) participate in the adjustment phase.

The intranasal treatment frequency will be reduced from that in the induction phase (twice weekly) to weekly for the first 4 weeks of the 12 weeks of this phase. After the first 4 weeks, the frequency of intranasal treatment sessions will be individualized to either once weekly or once every other week based on the severity of depressive symptoms. The dose of intranasal esketamine per session will remain unchanged from the dose at the end of the induction phase. One device will be used at each designated time point and each device contains 2 sprays. The intranasal devices containing esketamine deliver 14 mg per spray, for a total of 28 mg per individual device (i.e., 2 sprays). See, Table 11 for the intranasal administration schedule.

All subjects will continue taking the same oral antidepressant treatment (at the same dosage) that was initiated during the open-label induction phase.

TABLE 11

| | Time of Administration | | |
|---|---|---|---|
| | 0^{a} | 5 minutes | 10 minutes |
| | Intranasal Device | | |
| | First | Second | Third |
| Placebo | 1 spray of placebo | 1 spray of placebo | 1 spray of placebo |
| | to each nostril | to each nostril | to each nostril |
| Esketamine 56 mg | 1 spray of esketamine | 1 spray of esketamine | 1 spray of placebo |
| | to each nostril | to each nostril | to each nostril |
| Esketamine 84 mg | 1 spray of esketamine | 1 spray of esketamine | 1 spray of esketamine |
| | to each nostril | to each nostril | to each nostril |

For subjects in stable remission (MADRS total score 12 for the last 4 weeks of the maintenance phase) and those with stable response (≧50% reduction in the MADRS total score from baseline (Day 1 of induction phase; pre-randomization/prior to the first intranasal dose) in each of the last 4 weeks of the maintenance phase with at least 1 MADRS total score of >12 in these 4 weeks) at the end of this phase, the last visit of the adjustment phase serves as the baseline visit of the maintenance phase described below.

C. Maintenance Phase

At the end of the optimization phase, subjects in stable remission and those with stable response (but who are not in stable remission) continue into the maintenance phase. This phase will have a variable duration. One device will be used at each designated time point and each device contains 2 sprays. The intranasal devices containing esketamine deliver 14 mg per spray, for a total of 28 mg per individual device (i.e., 2 sprays). Table 12 describes the administration of intranasal study drug in this phase.

TABLE 12

| | Time of Administration | | |
|---|---|---|---|
| | 0^{a} | 5 minutes | 10 minutes |
| | Intranasal Device | | |
| | First | Second | Third |
| Placebo | 1 spray of placebo | 1 spray of placebo | 1 spray of placebo |
| | to each nostril | to each nostril | to each nostril |
| Esketamine 56 mg | 1 spray of esketamine | 1 spray of esketamine | 1 spray of placebo |
| | to each nostril | to each nostril | to each nostril |
| Esketamine 84 mg | 1 spray of esketamine | 1 spray of esketamine | 1 spray of esketamine |
| | to each nostril | to each nostril | to each nostril |

On Day 1 of this phase:

(i) Approximately 211 subjects in stable remission at the end of the optimization phase (after treatment with intranasal esketamine plus an oral antidepressant) will be randomized in a 1:1 ratio to either continue with intranasal esketamine (same dose) and the same oral antidepressant or to continue with the same oral antidepressant (active comparator) but switch to intranasal placebo.

(ii) Subjects with stable response (but who are not in stable remission) at the end of the optimization phase (after treatment with intranasal esketamine plus an oral antidepressant) will be randomized in a 1:1 ratio (using a separate randomization list) to either continue with intranasal esketamine (same dose) and the same oral antidepressant or to continue with the same oral antidepressant (active comparator) but switch to intranasal placebo (for a secondary efficacy analysis only).

The frequency of intranasal treatment sessions will be further individualized during the maintenance phase to once weekly or once every other week based on the severity of depressive symptoms, as assessed by the MADRS total score or another score as discussed above.

### Example 6

### Intranasal Esketamine Plus an Oral Antidepressant in Elderly Subjects with Treatment-Resistant Depression

This example is performed to evaluate the efficacy of switching elderly subjects with TRD from a prior antidepressant treatment to which they have not responded to flexibly dosed intranasal esketamine plus a newly initiated oral antidepressant compared with switching to a newly initiated oral antidepressant plus intranasal placebo, in improving depressive symptoms.

The study population will include elderly men and women, 65 years (inclusive) and older, who meet the Diagnostic and Statistical Manual of Mental Disorders (5th Edition; DSM-5) diagnostic criteria for single-episode MDD (if a single episode MDD, the duration must be ≧2 years) or recurrent MDD, without psychotic features, based upon clinical assessment.

A. Induction Phase

Subjects will be randomly assigned at a 1:1 ratio to receive double-blind intranasal treatment with either esketamine or placebo. In addition, subjects will simultaneously initiate a new, open-label oral antidepressant on Day 1 that will be continued for the duration of this phase. See, Table 13 for a summary for the below-noted administrations.

(i) Intranasal Study Medication

All subjects will self-administer the intranasal study drug (esketamine or placebo) at treatment sessions twice a week for 4 weeks at the study site. One device will be used at each time point and each intranasal device contains 2 sprays. The intranasal devices containing esketamine deliver 14 mg per spray, for a total of 28 mg per individual device (i.e., 2 sprays). The first treatment session will be on Day 1. Intranasal treatment sessions will not take place on consecutive days.

(a) On Day 1, all subjects randomized to intranasal esketamine (56 mg or 84 mg) will start with a dose of 28 mg.

(b) On Day 4, this dose will be increased to 56 mg.

(c) On Day 8, the dose may be increased to 84 mg or remain at 56 mg, as determined by the investigator based on efficacy and tolerability.

(d) On Day 11, the dose may be increased to 84 mg (if Day 8 dose was 56 mg), remain the same, or be reduced to 56 mg (if Day 8 dose was 84 mg), as determined by the investigator based on efficacy and tolerability.

(e) On Day 15, a dose reduction from 84 mg to 56 mg is permitted, if required for tolerability; no dose increase is permitted on Day 15.

(f) After Day 15, the dose must remain stable (unchanged).

TABLE 13

| | Time of Intranasal Device Administration | | |
|---|---|---|---|
| | 0^{a} | 5 minutes | 10 minutes |
| Intranasal | Intranasal Device | | |
| Treatment | 1st | 2nd | 3rd |
| Placebo for 28 mg | 1 spray of placebo to each | Not applicable | Not applicable |
| | nostril | | |
| Esketamine 28 mg | 1 spray of esketamine to | Not applicable | Not applicable |
| | each nostril | | |
| Placebo for 56 mg | 1 spray of placebo to each | 1 spray of placebo to | Not applicable |
| | nostril | each nostril | |
| Esketamine 56 mg | 1 spray of esketamine to | 1 spray of esketamine | Not applicable |
| | each nostril | to each nostril | |
| Placebo for 84 mg | 1 spray of placebo to each | 1 spray of placebo to | 1 spray of placebo to |
| | nostril | each nostril | each nostril |
| Esketamine 84 mg | 1 spray of esketamine to | 1 spray of esketamine | 1 spray of esketamine |
| | each nostril | to each nostril | to each nostril |

(ii) Oral Antidepressant Study Medication

Starting on Day 1, an open-label oral antidepressant treatment will be initiated in all subjects. The oral antidepressant will be 1 of 4 oral antidepressant medications (duloxetine, escitalopram, sertraline, or venlafaxine XR). The antidepressant medication will be assigned by the investigator based on review of MGH-ATRQ and relevant prior antidepressant medication information, and will be one that the subject has not previously had a nonresponse to in the current depressive episode, has not been previously intolerant to (lifetime)

Dosing of the oral antidepressant will begin on Day 1 and will follow the local prescribing information for the respective product (with adjustments to dosing made for the elderly if applicable), with a forced titration to the maximum tolerated dose. The titration schedule is provided in Table 14. If higher doses are not tolerated, a down-titration is permitted based on clinical judgment.

TABLE 14

| | Titration Schedule | | | |
|---|---|---|---|---|
| | | Week 2 | Week 3 | Week 4 |
| Oral | Week 1 | (Starting | (Starting | (Starting |
| Antidepressant | (Starting Day 1) | Day 8) | Day 15) | Day 22) |
| Duloxetine | 30 mg | 60 mg | 60 mg | 60 mg |
| Escitalopram | 10 mg | 10 mg | 10 mg | 10 mg |
| Sertraline | 25 mg | 50 mg | 100 mg | 150 mg |
| Venlafaxine XR | 37.5 mg | 75 mg | 150 mg | 150 mg |

All subjects will be provided with an additional 4-week supply of the oral antidepressant medication, to ensure that there is no interruption of oral antidepressant therapy.

### Example 7

This example is performed to evaluate the efficacy of intranasal esketamine plus an oral antidepressant in subjects with TRD. Subjects will include (i) direct-entry subjects or (ii) subjects from Example 6 (“transferred-entry subjects”). The transferred-entry subjects who are non-responders to the treatment protocol will be referred to as “transferred-entry non-responder subjects”. Transferred-entry subjects who are responders are referred to as “transferred-entry responder subjects”.

A. Induction Phase

In this phase, the subjects will self-administer open-label treatment with intranasal esketamine treatment twice a week for 4 weeks as a flexible dose regimen. Direct-entry subjects will initiate a new, open-label oral antidepressant. Transferred-entry subjects will continue the same oral antidepressant as they received in the induction phase of Example 6.

(i) Direct-Entry Subjects

Intranasal esketamine treatment will be self-administered, twice weekly for 4 weeks according to the schedules set forth in Table 15 for direct-entry subjects <65 years of age. One device will be used at each time point. All direct-entry subjects will initiate a new, open-label oral antidepressant on Day 1, which will be taken daily during the study. The oral antidepressant will be 1 of 4 oral antidepressant medications (duloxetine, escitalopram, sertraline, or venlafaxine XR).

TABLE 15

| Day | Dose (mg) | Dose Titration |
|---|---|---|
| 1 | 56 | |
| 4 | 56 or 84 | The dose may be increased to 84 mg or remain |
| | | at 56 mg |
| 8 | 56 or 84 | The dose may be increased to 84 mg |
| | | (if Day 4 dose was 56 mg), remain the same as |
| | | Day 4, or be reduced to 56 mg (if Day 4 dose was |
| | | 84 mg) |
| 11 | 56 or 84 | The dose may be increased to 84 mg (if |
| | | Day 8 dose was 56 mg), remain the same as on |
| | | Day 8, or be reduced to 56 mg (if Day 8 dose |
| | | was 84 mg) |
| 15 | 56 or 84 | A dose reduction from 84 mg to 56 mg is |
| | | permitted. If the dose is 56 mg on Day 11, no |
| | | dose increase is permitted on Day 15 |
| 18, 22, 25 | 56 or 84 | No dose increase from 56 mg is permitted |
| | | beyond Day 15. One additional dose down- |
| | | titration from 84 mg to 56 mg is permitted from |
| | | Day 15 until Day 25 |

Each intranasal device contains 2 sprays. The intranasal devices containing esketamine deliver 14 mg per spray, for a total of 28 mg per individual device, i.e., 2 sprays. See, Table 16 for the timing of the intranasal administration.

TABLE 16

| | Time of Administration | | |
|---|---|---|---|
| | 0 | 5 minutes | 10 minutes |
| Intranasal | Intranasal Device | | |
| Treatment | First | Second | Third |
| Esketamine 56 mg | 1 spray of esketamine | 1 spray of esketamine | No device required |
| | to each nostril | to each nostril | |
| Esketamine 84 mg | 1 spray of esketamine | 1 spray of esketamine | 1 spray of esketamine |
| | to each nostril | to each nostril | to each nostril |

(ii) Transferred-Entry Non-Responder Subjects

Subjects who do not respond to TRD treatment and are ≧65 years of age, including patients from Example 6 study, will participate in this example. See, Table 17 for the dosing regimen. These subjects will continue taking the same oral antidepressant at the same dose as taken in the last week of the double-blind induction phase of Example 6.

TABLE 17

| Day | Dose (mg) | Dose Titration Guidance |
|---|---|---|
| 1 | 28 | |
| 4 | 56 | |
| 8 | 56 or 84 | The dose may be increased to 84 mg or remain |
| | | at 56 mg |
| 11 | 56 or 84 | The dose may be increased to 84 mg (if Day 8 |
| | | dose was 56 mg), remain the same, or be |
| | | reduced to 56 mg (if Day 8 dose was 84 mg) |
| 15 | 56 or 84 | A dose reduction from 84 mg to 56 mg is |
| | | permitted. If the dose was 56 mg on Day 11, |
| | | no dose increase is permitted on Day 15. |
| 18, 22, 25 | 56 or 84 | No dose increase from 56 mg is |
| | | permitted beyond Day 15. If needed, one |
| | | additional dose down-titration from 84 mg |
| | | to 56 mg is permitted from Day 15 until Day 25. |

(iv) Oral Antidepressant

Dosing of the oral antidepressant will have a forced titration to the maximally tolerated dosage to ensure that the oral antidepressant is taken at an adequate dosage and duration for assessment of potential maintenance of effect. Titration schedules are provided in Table 18.

TABLE 18

| | Titration Schedule | | | |
|---|---|---|---|---|
| | Week 1 | Week 2 | | |
| Oral | (Start | (Start | Week 3 | Week 4 |
| Antidepressant | Day 1) | Day 8) | (Start Day 15) | (Start Day 22) |

Dosage (mg) Subjects <65 Years of Age

| | | | | |
|---|---|---|---|---|
| Duloxetine | 60 | 60 | 60 | 60 |
| Escitalopram | 10 | 20 | 20 | 20 |
| Sertraline | 50 | 100 | 150 | 150 |
| Venlafaxine XR | 75 | 150 | 225 | 225 |

Dosage (mg) Subjects ≧65 Years of Age

| | | | | |
|---|---|---|---|---|
| Duloxetine | 30 | 60 | 60 | 60 |
| Escitalopram | 10 | 10 | 10 | 10 |
| Sertraline | 25 | 50 | 100 | 150 |
| Venlafaxine XR | 37.5 | 75 | 150 | 150 |

B. Maintenance Phase

For all subjects, intranasal treatment session frequency will be reduced from that in the induction phase (twice weekly) to weekly for the first 4 weeks of the optimization/maintenance phase, i.e., Weeks 5-8. After the first 4 weeks, the frequency of intranasal treatment sessions will be adjusted to once weekly or once every other week based on the severity of depressive symptoms. Subjects will continue to take the same oral antidepressant (at the same dose) that they received at the end of the induction phase. Each intranasal device contains 2 sprays. The intranasal devices containing esketamine deliver 14 mg per spray, for a total of 28 mg per individual device, i.e., 2 sprays. See, Table 19 for the timing of the intranasal administration.

TABLE 19

| | Time of Administration | | |
|---|---|---|---|
| | 0^{a} | 5 minutes | 10 minutes |
| | Intranasal Device | | |
| | First | Second | Third |
| Esketamine 28 mg^{b} | 1 spray of esketamine | No device required | No device required |
| | to each nostril | | |
| Esketamine 56 mg | 1 spray of esketamine | 1 spray of esketamine | No device required. |
| | to each nostril | to each nostril | |
| Esketamine 84 mg | 1 spray of esketamine | 1 spray of esketamine | 1 spray of esketamine |
| | to each nostril | to each nostril | to each nostril |

^{a}Time 0 is defined as the time of administration of the first intranasal spray to one nostril from the first intranasal device
^{b}28 mg dose will be administered only for transferred-entry responder subjects, at Week 5.

(i) Direct-Entry Subjects

The direct-entry patients will include adult men and women, years of age who completed the induction phase noted above. Subjects will continue to receive the same intranasal treatment from the open-label induction phase.

(ii) Transferred-Entry Responder and Non-Responder Subjects

The transferred-entry responder and non-responder subjects are elderly (≧65 years old) subjects who have completed the induction phase noted above. These subjects will all start intranasal esketamine with an initial dose of 28 mg (Week 5; Study Day 32) and have their dose adjusted over the following 3 weeks of the maintenance phase. See, Table 20.

TABLE 20

| Week | Dose (mg) | Dose Titration Guidance |
|---|---|---|
| 5 | 28 | |
| 6 | 56 | |
| 7 | 56 or 84 | may be increased to 84 mg or remain at 56 mg |
| 8 | 56 or 84 | a dose reduction from 84 mg to 56 mg (if Week 7 dose |
| | | was 84 mg) is permitted; If dose was 56 mg in |
| | | Week 7, no dose increase is permitted in Week 8 |
| 9+ | 56 or 84 | one additional dose down-titration is allowed |

The disclosures of each patent, patent application, and publication cited or described in this document are hereby incorporated herein by reference, in its entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

We claim:

1. A method of treating depression in a patient, said method comprising:
(a) administering an effective amount of esketamine to said patient at a given frequency and during an induction phase of defined duration; and
(b) administering an effective amount of esketamine to said patient less frequently during a subsequent maintenance phase of defined duration.

2. The method of claim 1, wherein said esketamine is administered during said induction phase at a frequency of at least once weekly for at least about 1 week; and said esketamine is administered during said maintenance phase at a frequency of no more often than every other day.

3. The method of claim 1 or 2, wherein said induction phase is about 1 to about 12 weeks.

4. The method of claim 1 or 2, wherein said induction phase is about 2 to about 8 weeks.

5. The method of claim 1 or 2, wherein said induction phase is at least about 2 weeks.

6. The method of claim 1 or 2, wherein said induction phase is at least about 3 weeks.

7. The method of claim 1 or 2, wherein said induction phase is at least about 4 weeks.

8. The method of claim 1 or 2, wherein said maintenance phase further comprises adjusting the frequency of administration of said esketamine.

9. The method of claim 1 or 2, wherein said frequency of administration of said esketamine is increased in said maintenance phase.

10. The method of claim 1 or 2, wherein said frequency of administration of said esketamine is decreased in said maintenance phase.

11. The method of any one of claims 1 to 10, wherein said effective amount in each dose is about 10 to about 200 mg.

12. The method of claim 11, wherein said effective amount in each dose is about 14 mg.

13. The method of claim 11, wherein said effective amount in each dose is about 28 mg.

14. The method of claim 11, wherein said effective amount in each dose is about 56 mg.

15. The method of claim 11, wherein said effective amount in each dose is about 84 mg.

16. The method of claim 1 or 2, wherein said esketamine is administered during said induction phase, said maintenance phase, or any combination thereof at the lowest dosing frequency at which an esketamine response is observed in said patient.

17. The method of claim 1 or 2, wherein said esketamine is administered at the lowest dosage amount at which an esketamine response is observed in said patient.

18. The method of claim 1 or 2, wherein said esketamine is administered non-orally during said induction phase.

19. The method of claim 18, wherein said esketamine is administered intravenously, intranasally, intramuscularly, subcutaneously, transdermally, buccally, or rectally during said induction phase.

20. The method of claim 19, wherein said esketamine is administered intranasally during said induction phase.

21. The method of claim 1 or 2, wherein said esketamine is administered orally during said maintenance phase.

22. The method of claim 1 or 2, wherein said esketamine is administered orally during said induction phase.

23. The method of claim 1 or 2, wherein a placebo is administered on any day during said induction phase or maintenance phase on which said esketamine is not administered.

24. The method of any one of claims 1 to 23, wherein said depression is treatment resistant depression.

25. The method of any one of claims 1 to 24, wherein the dosage of esketamine is optimized for a patient having or being predisposed to depression.

26. The method of any one of claims 1 to 25, wherein the effect of said esketamine is analyzed by evaluating the depression of said subject.

27. A kit for administering esketamine to a patient in need thereof, said kit comprising:
(a) a first dosage unit comprising an effective amount of esketamine for administration to a patient at a given frequency in an induction phase of at least about 1 week; and
(b) a second dosage unit comprising an effective amount of esketamine for administration to said patient less frequently than in said induction phase of a defined duration in a maintenance phase;
wherein said second dosage unit is administered to said patient after said induction phase.

28. A kit for administering esketamine to a patient in need thereof, said kit comprising:
(a) a first dosage unit comprising an effective amount of esketamine for administration to a patient at a frequency of at least once weekly in an induction phase of at least about 1 week; and
(b) a second dosage unit comprising an effective amount of esketamine for administration to said patient at a frequently less than the frequency in said induction phase in a maintenance phase;
wherein said second dosage unit is administered to said patient after said induction phase.

29. The kit of claim 27 or 28, wherein said first dosage unit is a non-oral dosage unit.

30. The kit of claim 29, wherein said non-oral dosage unit is an intravenous, intranasal, intramuscular, subcutaneous, transdermal, buccal, or rectal dosage unit.

31. The kit of claim 30, wherein said first dosage unit is an intranasal dosage unit.

32. The kit of claim 27 or 28, wherein said first dosage unit is an oral dosage unit.

33. The kit of claim 27 or 28, wherein said second dosage unit is an oral dosage unit.

34. The kit of claim 27 or 28, further comprising a means for evaluating said patient's depression.

35. The kit of claim 34, further comprising a depression scale rating scale questionnaire.
